(19) United States
(12) Patent Application Publication
(10) Pub. No.: US 2022/0047833 A1
(43) Pub. Date: Feb. 17, 2022

(54) TRACHEOSTOMY COLLAR

(71) Applicant: AMBER NICHOLS FOWLER, Santa Barbara, CA (US)

(72) Inventor: AMBER NICHOLS FOWLER, Santa Barbara, CA (US)

(21) Appl. No.: 16/991,785

(22) Filed: Aug. 12, 2020

(51) Int. Cl. A61M 16/04

(52) U.S. Cl. CPC A61M 16/0497; A61M 16/0465

(57) ABSTRACT

A reusable quick release tracheostomy collar or band is provided. The tracheostomy collar or band comprises three quick releases so that the tracheostomy collar or band can be quickly removed or freed up such that a tracheostomy tube may be quickly removed. The tracheostomy collar or band includes two chain portions. Each chain portion carries a hook to attach to flanges on the tracheostomy tube. Each hook engages an aperture in a corresponding one of the flanges. In addition, the two chains are connected together at their opposite ends with a hook and catch. The tracheostomy collar or band is of hypoallergenic material.

### FIELD OF THE INVENTION

This invention relates to a tracheostomy collar for holding a tracheostomy tube securely in place on the neck of a patient.

### BACKGROUND OF THE INVENTION

Some people are born with a medical condition that requires them to have a tracheotomy. Others have tracheotomy operations due to a specific medical need.

Tracheotomy is a surgical procedure in which an incision, called a tracheostomy, is made into the trachea through the neck tissues that forms a temporary or permanent opening, called a stoma. A tracheostomy tube is inserted through the opening to allow passage of air and removal of secretions. The tracheostomy tube allows air to flow through the tube and into the patient's lungs and further allows passage of other medical instruments into the trachea and connected organs.

One such medical condition, known as a Congenital Diaphragmatic Hernia or CDH, is a defect in which there is a hole in the diaphragm which allows the intestines, stomach and other organs to enter the chest cavity impairing normal lung development and impeding their ability to breathe. A tracheotomy is performed and a tracheostomy tube is placed in the stoma in the patient's neck. The tracheostomy tube is coupled to a ventilator that is necessary for the patient to stay alive.

Once the tracheostomy tube has been inserted it is important that the position of the tube be stabilized such that it is not inadvertently withdrawn from the trachea, and such that the tube is otherwise positioned to function properly. Tracheostomy tubes typically have a laterally extending flange surface configured to extend parallel to the front portion of the patient's neck when the tube is installed. The flange generally has two slots for connecting with a tracheostomy collar or band that encircles the patient's neck to hold the tracheostomy tube securely in place.

Typical prior art tracheostomy collars consist of foam.

Foam tracheostomy collars absorb liquid and secretions. Unfortunately, foam tracheostomy collars cause skin irritation and breakdown and may lead to infection. Accordingly, foam tracheostomy collars must be replaced at least once a day and sometimes more depending on the amount of secretions, liquid or sweat absorbed.

The cost of prior art foam bands used across the world cost ranges from $1.50 to $3.50 per band. One would typically use at least 1 or 2 bands per day. The result is a cost of $45 to $210 per month. This cost does not include going to the doctor to get a prescription when the patient gets an infection or the amount of money you spend to care for the surrounding skin. Although foam bands come in different sizes typically it is still required to cut the band in order to fit the patient's neck size. In addition it is necessary to place a prescription moisture barrier medication on it. Most insurance companies, including my nephews, will only supply one band per day and some would supply only one band per week. The result is the patient is forced to reuse the band intended for single use only. Reusing a medical device intended for single use only leads to germs, increased risk of infection and causes the band itself to breakdown.

It is highly desirable to provide a tracheostomy collar that avoids the problems of prior art tracheostomy collars.

### SUMMARY OF THE INVENTION

A reusable quick release tracheostomy collar or band is provided. The tracheostomy collar provides improved material and methods for its use that overcomes shortcomings of prior tracheostomy collars.

In accordance with the principles of the invention, an embodiment of a tracheostomy collar for securely stabilizing a tracheostomy tube in place on a patient's neck is provided. The tracheostomy tube comprises a first flange having a first aperture and a second flange having a second aperture. The collar comprises a first chain portion and a second chain portion. The first chain portion has a first end and a second end and carries a hook on the first end. The first hook is configured to removably engage the first aperture to removably connect the first chain portion to the first flange and to provide a first quick release connection. The second chain portion has a third end and a fourth end and carries a second hook on the third end. The second hook is configured to removably engage the second aperture to removably connect the second chain portion to the second flange and to provide a second quick release connection. The first chain portion second end carries a third hook and the second chain portion fourth end carries a hook receiving member. The third hook and the hook receiving member are configured so that the third hook removably engages the hook receiving member to removably secure the first chain portion to the second chain portion with a first quick release connection to hold the tracheostomy tube in place when the first hook engages the first aperture to provide a second quick release connection and the second hook engages the second aperture and to provide a third quick release connection.

In an embodiment of a tracheostomy collar, each of the first hook, the second hook, and the third hook has a rounded end.

In an embodiment of a tracheostomy, collar the hook receiving member comprises a clasp.

In an embodiment of a tracheostomy collar, each of the first chain portion, the second chain portion, the first hook, the second hook, the third hook, and the hook receiving member comprise hypoallergenic material.

In an embodiment of a tracheostomy collar the hypoallergenic material may comprise sterling silver.

In another embodiment, a tracheostomy collar for securely stabilizing a tracheostomy tube in place on a patient's neck is provided. The tracheostomy tube has one or more flanges. The collar comprises a chain releasably connectable to the one or more flanges to hold the tracheostomy tube in place on the patient's neck. The chain comprises at least two quick releases for releasing the collar from the patient's neck. Each of the two releases provides a quick release capability to quickly release the collar from the patient's neck.

In another embodiment, the chain may comprise hypoallergenic material.

The hypoallergenic material may comprise sterling silver.

In the another embodiment, the at least two releases may comprise a first hook carried on one end of the chain to engage the at least one of said one or more corresponding apertures, a second hook carried on another end of said chain to engage at least one of the one or more corresponding apertures, and a clasp disposed on said chain intermediate the one end and the another end. The clasp removably couples a first portion of the chain with a second portion of the chain. The first portion carries the first hook and the second portion carries the second hook.

In another embodiment, the clasp may comprise a hook carried on the first portion and a hook receiving member carried on the second portion

In another embodiment, at least one of the at least three releases may comprise mating magnetic catches.

In another embodiment, the one or more flanges may comprise a first flange and a second flange disposed on opposite sides of the tracheostomy tube.

In another embodiment, the at least two releases may comprise a first release apparatus releasably coupleable to the first flange and a second release apparatus releasably coupleable to the second flange.

In another embodiment, the chain may comprise a first chain portion having a first end carrying a first release and a second chain portion having a first end carrying a second release. The first chain portion may carry a first quick release catch member on a second end and said second chain portion may carry a second quick release catch member on a second end. The first quick release catch member and the second quick release catch member are adapted to releasably engage each other to couple the first chain portion with the second chain portion.

In yet another embodiment, a tracheostomy collar is provided for securely stabilizing a tracheostomy tube on a patient's neck to provide protection from undue discomfort. The tracheostomy tube is a type comprising first and second flanges; each of the flanges has a corresponding aperture to which the collar is releasably securable. The collar comprises a hypoallergenic chain assembly sized to secure the tracheostomy tube on the patient's neck. The chain assembly comprises a first chain portion coupled to the first flange aperture and a second chain portion coupled to the second chain portion. A first quick release catch assembly is carried on the first chain portion and a second quick release portion carried on the second chain portion. The first quick release portion and the second quick release portion are adapted to connect to each other to secure the tracheostomy collar on the patient's neck and to provide a quick release for removal of the tracheostomy collar and the tracheostomy tube.

In another embodiment, the first chain portion may comprise a first quick release connecting member to provide a quick release coupling to the first flange. The second chain portion may comprise a second quick release connecting member to provide a quick release coupling to the second flange.

In another embodiment, the first quick release connecting member may comprise a first hook to releasably engage the first aperture, and the second quick release connecting member may comprise a second hook to releasably engage the second aperture.

In another embodiment, the hypoallergenic chain assembly may comprise sterling silver.

In the another embodiment, the first quick release catch assembly may comprise a hook, and the second quick release catch assembly may comprise a catch to releasably receive the hook to provide a quick release coupling.

In the another embodiment, the first chain portion and said second chain portion may be sized to place the first quick release catch assembly and the second quick release assembly at the rear neck portion of the patient's neck when the first quick release catch assembly is coupled to the second quick release assembly.

### BRIEF DESCRIPTION OF THE DRAWING

The invention will be better understood from a reading of the following detailed description of embodiments of the invention in conjunction with the drawing figures in which like reference designators are used to identify like elements and in which:

FIG. 1 illustrates an embodiment of tracheostomy collar in accordance with the invention in use on a patient's neck;

FIG. 2 is a first view of a portion of the tracheostomy collar and tracheostomy tube of FIG. 1;

FIG. 3 is a first view of a portion of the tracheostomy collar and tracheostomy tube of FIG. 1;

FIG. 4 illustrates the tracheostomy collar of FIG. 1 apart from the tracheostomy tube;

FIG. 5 illustrates a portion of the tracheostomy collar of FIG. 4;

FIG. 6 illustrates the portion of the tracheostomy collar of FIG. 5; and

FIG. 7 is a further detailed portion of the tracheostomy collar of FIG. 5.

### DETAILED DESCRIPTION

I created and invented a hypo-allergenic band or collar for my nephew. The band or collar consists of sterling silver that fastens to a tracheostomy tube on both sides and has a clasp on the back. The band or collar is provided with three releases to facilitate quick removal in case of an emergency. The hypoallergenic band or collar is practical and cost effective. My band or collar does not cause any skin breakdown or infections and it is stylish. This band does not have to be changed daily or when wet. My nephew wears the same band all the time.

As a result of using the band or collar that I have invented, my nephew has not had any skin breakdown or infection. Also, it has saved time, money, and most importantly it has made for a pain free and infection free child—which is what everyone desires. My band or collar can be sized for an adult as well.

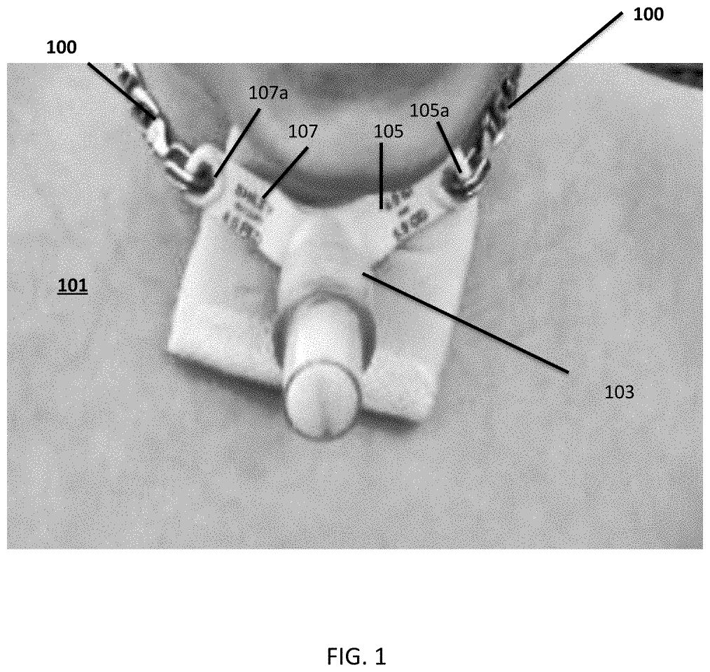

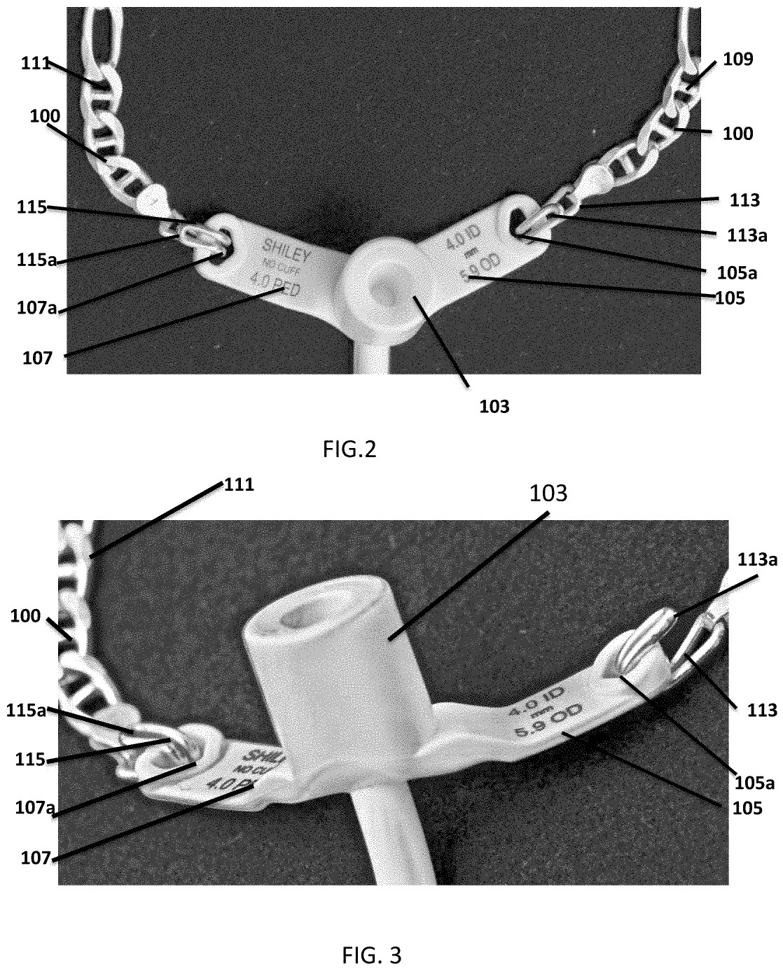

Turning now to FIG. 1, an embodiment of a tracheostomy collar or band 100 in accordance with the principles of my invention is shown while being worn by a patient 101 Tracheostomy band 100 secures a tracheostomy tube 103 on patient 101. Although the image of FIG. 1 is a frontal view of the patient 101, it is to be understood that collar 100 is releasably fastened at the back of the neck of patient 101 as will be become clear from other ones of the drawing figures. Tracheostomy tube 103 has flanges 105, 107. Each flange 105, 107 carries a corresponding aperture 105a, 107a, respectively, that are shown with greater clarity in FIGS. 2 and 3.

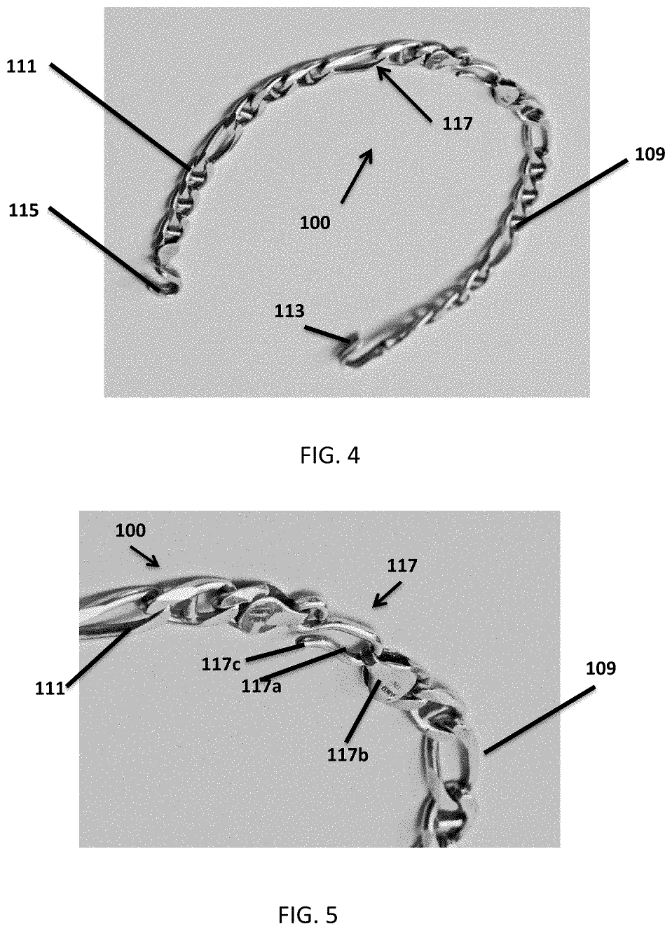

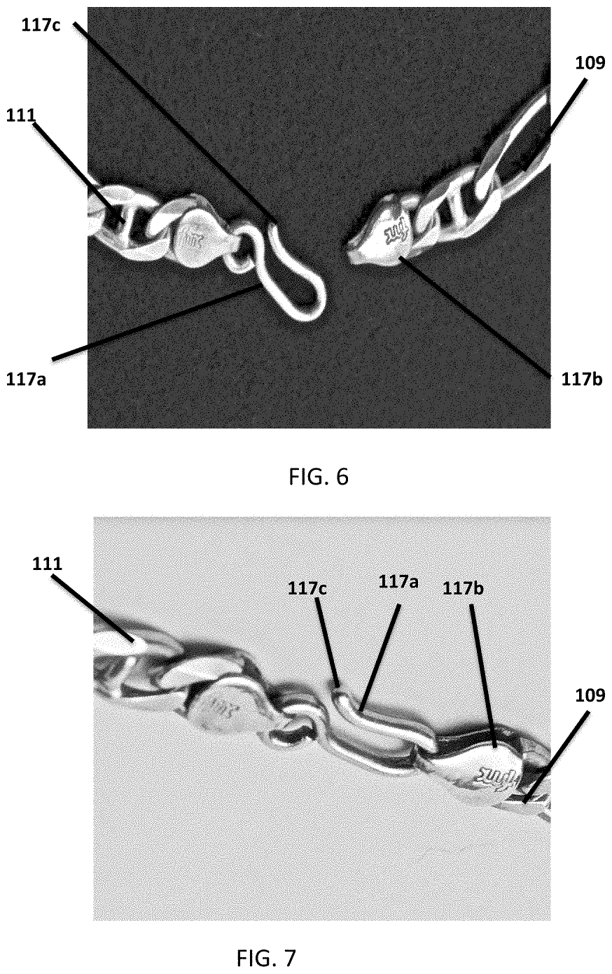

In FIGS. 2 and 3 tracheostomy collar or band 100 and tracheostomy tube 103 are shown apart from patient 101 so that the various features and details of the embodiment are more easily seen. Still further, FIGS. 4-7 show only tracheostomy collar or band 100 or portions thereof apart from tracheostomy tube 103.

As seen in the various figures, tracheostomy collar or band 100 comprises two sterling silver chain portions 109, 111 each comprising a plurality of sterling silver chain links. The chain links are decorative in appearance, creating an attractive as well as functional tracheostomy collar or band. It will be apparent to those skilled in the art that different chains may be used having different chain links. It will also be apparent to those skilled in the art that although the embodiment shown in the drawing figures utilizes sterling silver material to achieve a structure that is hypoallergenic, other hypoallergenic materials or combinations of hypoallergenic materials may be utilized.

Three quick release attachments are provided on tracheostomy collar or band 100 to provide for quick removal of tracheostomy collar or band 100 and if necessary removal of tracheostomy tube 103.

FIGS. 2 and 3 more clearly show two quick release couplings 113, 115 of tracheostomy band or collar 100 coupled to tracheostomy tube 103. Each quick release coupling 113, 115 comprises a hook 113a, 115a. Hooks 113a, 115a releasably engage tracheostomy tube 103 flanges 105, 107. Hook 113a engages flange 105 through aperture 105a, and hook 115a engages flange 107 through aperture 107a. Each hook 113a, 115a has a corresponding rounded end 113b, 115b to facilitate insertion into apertures 105a, 107a. Hooks 113a, 115a are sized to facilitate easy and quick insertion into apertures 105a, 107a as well as easy and quick removal from apertures 105a, 107a thereby facilitating fast attachment to tracheostomy tube 103 as well as fast removal therefrom. Each hook 113a, 115a is coupled to respective chain portions 109, 111 with attachment element 109a, 111a. It will be appreciated by those skilled in the art that other hook configurations may be utilized and different attachment elements may also be utilized.

FIG. 4 shows the entirety of tracheostomy collar or band 100 without tracheostomy tube 103. FIGS. 5-7 show in close up details of third quick release 117. In the embodiment, third quick release 117 is disposed on tracheostomy collar or band 100 such that when being worn on patient 101, it is located at the rear of the neck of patient 101.

Third quick release 117 is a clasp that comprises a hook 117a carried on chain portion 111 and a hook receiving member or catch 117b carried on chain portion 109. Hook 117a has a rounded end 117c that facilitates insertion into catch 117b. By providing a hook 117a and catch 117b release chain portions 111, 109 may be quickly released or detached from each other.

In other embodiments one or more of the quick releases 113, 115, 117 may be replaced with other quick release apparatus. One such other quick release apparatus may comprise correlated magnets.

The invention has been described in terms of various embodiments. It will be appreciated by those skilled in the art that the scope of my invention is not limited to such embodiments and that various modifications and changes may be made to the various embodiments without departing from the spirit or scope of my invention. It is intended that my invention be limited in scope only by the claims appended hereto.

1. A reusable, quick release tracheostomy collar for securely stabilizing a tracheostomy tube in place on a patient's neck, said tracheostomy tube comprising a first flange comprising a first aperture and a second flange comprising a second aperture, said collar comprising:
a first chain portion having a first end and a second end;
a first hook carried by said first end, said first hook configured to removably engage said first aperture to removably connect said first chain portion to said first flange and to provide a first quick release connection;
a second chain portion having a third end and a fourth end;
a second hook carried by said third end, said second hook configured to removably engage said second aperture to removably connect said second chain portion to said second flange and to provide a second quick release connection;
a third hook carried on said second end;
a hook receiving member carried on said fourth end;
said third hook and said hook receiving member configured such that said third hook removably engages said hook receiving member to removably secure said first chain portion to said second chain portion to hold said tracheostomy tube in place with a first quick release connection when said first hook engages said first aperture to provide a second quick release connection and said second hook engages said second aperture and to provide a third quick release connection.

2. A reusable, quick release, tracheostomy collar in accordance with claim 1, wherein:
each of said first hook, said second hook and said third hook comprises a rounded end.

3. A reusable, quick release, tracheostomy collar in accordance with claim 2 wherein:
said hook receiving member comprises a clasp.

4. A reusable, quick release, tracheostomy collar in accordance with claim 1 wherein:
each of said first chain portion, said second chain portion, said first hook, said second hook, said third hook, and said hook receiving member comprise hypoallergenic material.

5. A reusable, quick release, tracheostomy collar in accordance with claim 4 wherein:
said hypoallergenic material comprises sterling silver.

6. A reusable, quick release, tracheostomy collar for securely stabilizing a tracheostomy tube in place on a patient's neck, said tracheostomy tube comprising one or more flanges, said collar comprising:
a chain releasably connectable to said one or more flanges to hold said tracheostomy tube in place on said patient's neck; and
said chain comprising at least two releases for releasing said collar from said patient's neck, each of said two releases providing a quick release capability quickly release said collar from said patient's neck.

7. A reusable, quick release, reusable, quick release, tracheostomy collar in accordance with claim 6, wherein:
said chain comprises hypoallergenic material.

8. A reusable, quick release, tracheostomy collar in accordance with claim 7 wherein:
said hypoallergenic material comprises sterling silver.

9. A reusable, quick release, tracheostomy collar in accordance with claim 6, wherein:
said at least two releases comprise a first hook carried on one end of said chain to engage said at least one of said one or more corresponding apertures, a second hook carried on another end of said chain to engage at least one of said one or more corresponding apertures, and a clasp disposed on said chain intermediate said one end and said another end, said clasp removably coupling a first portion of said chain with a second portion of said chain, said first portion carrying said first hook and said second portion carrying said second hook.

10. A reusable, quick release, tracheostomy collar in accordance with claim 9, wherein:
said clasp comprises a hook carried on said first portion and a hook receiving member carried on said second portion

11. A reusable, quick release, tracheostomy collar in accordance with claim 6, wherein:
at least one of said at least two releases comprises mating magnetic catches.

12. A reusable, quick release, tracheostomy collar in accordance with claim 6, wherein:
said one or more flanges comprises a first flange and a second flange disposed on opposite sides of said tracheostomy tube.

13. A reusable, quick release, tracheostomy collar in accordance with claim 12, wherein:
said at least two releases comprise a first release apparatus releasably coupleable to said first flange and a second release apparatus releasably coupleable to said second flange.

14. A reusable, quick release, tracheostomy collar in accordance with claim 6, wherein:
said chain comprises a first chain portion having a first end carrying a first release, a second chain portion having a first end carrying a second release, said first chain portion carrying a first quick release catch member on a second end and said second chain portion carrying a second quick release catch member on a second end, said first quick release catch member and said second quick release catch member adapted to releasably engage each other to couple said first chain portion with said second chain portion.

15. A reusable, quick release, tracheostomy collar for securely stabilizing a tracheostomy tube on a patient's neck to provide protection from undue discomfort, said tracheostomy tube comprising first and second flanges, each of said first and second flanges comprising a corresponding aperture to which the collar is releasably securable, said collar comprising:
a hypoallergenic chain assembly sized to secure said tracheostomy tube on said patients neck, said chain assembly comprising a first chain portion coupled to said first flange aperture and a second chain portion coupled to said second chain portion, a first quick release catch assembly carried on said first chain portion and a second quick release portion carried on said second chain portion, said first quick release portion and said second quick release portion adapted to connect to each other to secure said tracheostomy collar on said patient's neck and to provide a quick release for removal of said tracheostomy collar and said tracheostomy tube.

16. A reusable, quick release, tracheostomy collar in accordance with claim 15, wherein:
said first chain portion comprises a first quick release connecting member to provide a quick release coupling to said first flange; and
said second chain portion comprise a second quick release connecting member to provide a quick release coupling to said second flange.

17. A reusable, quick release, tracheostomy collar in accordance with claim 16, wherein:
said first quick release connecting member comprises a first hook to releasably engage said first aperture; and
said second quick release connecting member comprises a second hook to releasably engage said second aperture.

18. A reusable, quick release, tracheostomy collar in accordance with claim 17, wherein:
said hypoallergenic chain assembly comprises sterling silver.

19. A reusable, quick release, tracheostomy collar in accordance with claim 16, wherein:
said first quick release catch assembly comprises a hook; and
said second quick release catch assembly comprises a catch to releasably receive said hook to provide a quick release coupling.

20. A reusable, quick release, tracheostomy collar in accordance with claim 16, wherein:
said first chain portion and said second chain portion are sized to place said first quick release catch assembly and said second quick release assembly at the rear neck portion of said patient's neck when said first quick release catch assembly is coupled to said second quick release assembly.
